(19) United States
(12) Patent Application Publication
(10) Pub. No.: US 2020/0154988 A1
(43) Pub. Date: May 21, 2020

(54) IMAGING DEVICE AND IMAGING METHOD

(71) Applicant: SONY CORPORATION, TOKYO (JP)

(72) Inventors: TAKANORI FUKAZAWA, KANAGAWA (JP); GORO FUJITA, KANAGAWA (JP); KOICHIRO KISHIMA, KANAGAWA (JP); KAZUKI IKESHITA, SAITAMA (JP); SHUN SASAKI, TOKYO (JP)

(21) Appl. No.: 16/610,586

(22) PCT Filed: Apr. 18, 2018
(86) PCT No.: PCT/JP2018/015947

§ 371 Date: Nov. 4, 2019

(30) Foreign Application Priority Data
May 12, 2017 (JP) 2017-095564

(51) Int. Cl. A61B 1/045; A61B 5/00; A61B 1/07; G02B 23/26; A61B 1/00

(52) U.S. Cl. CPC A61B 1/045; A61B 5/0059; A61B 1/00064; G02B 23/26; A61B 1/07

(57) ABSTRACT

An imaging device according to an embodiment of the present technology includes a first polarization section, a second polarization section, a rotation control section, and a generation section. The first polarization section irradiates a body tissue with polarization light of a first polarization direction. The second polarization section extracts a polarization component of a second polarization direction that intersects with the first polarization direction, from beams of reflection light that are the polarization light reflected by the body tissue. The rotation control section that rotates each of the first polarization direction and the second polarization direction while maintaining an intersection angle between the first polarization direction and the second polarization direction. The generation section that generates an image signal of the body tissue on the basis of the polarization component of the reflection light extracted by the second polarization section in accordance with rotation operation performed by the rotation control section.

### TECHNICAL FIELD

The present technology relates to an imaging device and an imaging method that are applicable to observation of a body tissue or the like.

### BACKGROUND ART

Conventionally, technologies of observing a body tissue irradiated with polarized light have been developed. For example, Patent Literature 1 describes a polarization image measurement display system that displays a polarization property of a site of lesion or the like. According to Patent Literature 1, an imaging section captures 16 or more light intensity polarization images in different polarization states. A polarization conversion process section calculates a Mueller matrix of 4 rows×4 columns on the basis of the light intensity polarization images, and generates a polarization property image that shows a polarization property such as a depolarization ratio of a sample or a polarization ratio of light by using the Mueller matrix. When a combination of such polarization property images is displayed, it is possible for a doctor to identify presence or absence of a collagen fiber or the like, and this makes it possible to support diagnosis such as a degree of invasiveness of intramucosal cancer (see paragraphs [0022], [0044] to [0046], [0094], FIG. 7, FIG. 15, and the like of Patent Literature 1).

### CITATION LIST

### Patent Literature

Patent Literature 1: JP 2015-33587A

### DISCLOSURE OF INVENTION

### Technical Problem

Such body tissue observation using polarization is expected to be applied to various situations such as surgery, medical diagnosis, and the like. Technologies capable of sufficiently supporting observation of body tissues have been desired.

In view of the circumstances as described above, it is an object of the present technology to provide an imaging device and an imaging method that are capable of sufficiently observing body tissues.

### Solution to Problem

In order to accomplish the above-mentioned object, an imaging device according to an embodiment of the present technology includes a first polarization section, a second polarization section, a rotation control section, and a generation section.

The first polarization section irradiates a body tissue with polarization light of a first polarization direction.

The second polarization section extracts a polarization component of a second polarization direction that intersects with the first polarization direction, from beams of reflection light that are the polarization light reflected by the body tissue.

The rotation control section rotates each of the first polarization direction and the second polarization direction while maintaining an intersection angle between the first polarization direction and the second polarization direction.

The generation section generates an image signal of the body tissue on the basis of the polarization component of the reflection light extracted by the second polarization section in accordance with rotation operation performed by the rotation control section.

the imaging device irradiates a body tissue with polarization light of the first polarization direction. Among beams of reflection light reflected by the body tissue, a polarization component of a second polarization direction that intersects with the first polarization direction is extracted. The first polarization direction and the second polarization direction are rotated while the intersection angle is maintained, and an image signal of the body tissue is generated on the basis of the extracted polarization component of the reflection light in accordance with the rotation operation. By using the image signal generated through the rotation operation, it is possible to sufficiently support observation of the body tissue.

The first polarization section may include a first polarization element that polarizes at least part of illumination light emitted from a light source, in the first polarization direction. In this case, the second polarization section may include a second polarization element that extracts the polarization component of the second polarization direction.

For example, this makes it possible to easily generate polarization light of the first polarization direction on the basis of illumination light emitted from various kinds of light sources.

A desirable intersection angle is 90°, but the intersection angle may be an angle in a range of 90°±2°.

Accordingly, the first polarization direction and the second polarization direction become a substantially crossed nicols state. As a result, it is possible to accurately detect change in polarization directions or the like with regard to the body tissue, and it is possible to sufficiently support observation of the body tissue.

The rotation control section may rotate the first polarization direction and the second polarization direction in synchronization with each other.

Therefore, for example, it is possible to shorten time it takes to generate an image signal through substantially simultaneous rotation of the first and second polarization directions. As a result, it is possible to observe the body tissue in real time.

The generation section may generate a first image signal in the case where each of the first polarization direction and the second polarization direction is in a predetermined state, and generate a second image signal in the case where the rotation control section rotates each of the first polarization direction and the second polarization direction in the predetermined state by a predetermined angle.

This makes it possible to easily detect parts or the like having different optical anisotropies in a body tissue on the basis of the first and second image signals, and this makes it possible to sufficiently support observation of the body tissue.

A desirable predetermined angle is 45°, but the predetermined angle may be an angle in a range of 45°±22.5°.

This makes it possible to accurately detect change in amount of reflected light or the like before and after rotation of the first and second polarization directions. As a result, it is possible to accurately detect the body tissue.

The imaging device according may further include an analysis section that analyzes each of the first image signal and the second image signal.

For example, it is possible to accurately detect parts or the like having different optical anisotropies in the body tissue on the basis of the first and second image signals. This makes it possible to sufficiently support observation of the body tissue.

The first image signal may include a plurality of first pixel signals each including luminance information. In this case, the second image signal may include a plurality of second pixel signals each including luminance information. In addition, the analysis section may calculate luminance differences between the first pixel signals and the second pixels signals.

For example, this makes it possible to detect change in amount of reflected light or the like for each pixel before and after rotation of the first and second polarization directions, and this makes it possible to accurately detect parts or the like having different optical anisotropies.

The analysis section may extract a part having the luminance difference higher than a predetermined threshold.

This makes it possible to extract positions and sizes of the parts having different optical anisotropies, for example.

The analysis section may generate an emphasis image in which the extracted part is emphasized.

For example, by using the emphasis image, it is possible to easily recognize the positions and sizes of the parts having different optical anisotropies.

The analysis section may output the emphasis image as an intraoperative image.

This makes it possible to observe the body tissue in detail during surgery or inspection, and this makes it possible to sufficiently support observation of the body tissue.

Each of the first polarization element and the second polarization element may be configured to be attachable and detachable.

By detaching each of the polarization elements, it is possible to carry out observation in a bright field without polarizing illumination light, for example. This makes it possible to carry out observation in accordance with types or the like of body tissues, and this results in improvement in convenience.

The imaging device may be configured as an endoscope or a microscope.

It is possible to sufficiently support observation of body tissues during inspection or the like using an endoscope or a microscope.

The rotation control section may be capable of setting the first polarization direction and the second polarization direction to be substantially parallel to each other.

This makes it possible to brightly display body tissues. Therefore, for example, it is possible to switch visibility of a body tissue in accordance with situations. For example, in the case where large halo is observed through reflection of a subject by a mirror, it is possible to suppress the halo by adjusting the first and second polarization directions.

An imaging method according to an embodiment of the present technology is an imaging method that is executed by a computer system, and the imaging method includes irradiating a body tissue with polarization light of a first polarization direction.

A polarization component of a second polarization direction that intersects with the first polarization direction is extracted from beams of reflection light that are the polarization light reflected by the body tissue.

Each of the first polarization direction and the second polarization direction is rotated while maintaining an intersection angle between the first polarization direction and the second polarization direction.

An image signal of the body tissue is generated on the basis of the extracted polarization component of the reflection light in accordance with rotation operation performed by the rotation control section.

### Advantageous Effects of Invention

As described above, it is possible to sufficiently support observation of body tissues. Note that the effects described herein are not necessarily limited and may be any of the effects described in the present disclosure.

### BRIEF DESCRIPTION OF DRAWINGS

FIG. 1 is a diagram schematically illustrating a configuration example of an endoscopic device that is an imaging device according to a first embodiment.

FIG. 2 is a schematic diagram illustrating an example of reflection by an observation target.

FIG. 3 is a diagram illustrating specific examples of specular reflection.

FIG. 4 is a schematic diagram illustrating examples of reflection caused inside an observation target.

FIG. 5 is a graph showing intensity of transmitted light that has passed through a second polarization element in the case of carrying out crossed nicols observation of an anisotropic body.

FIG. 6 is a flowchart illustrating an example of observation of a body tissue.

FIG. 7 is a diagram illustrating an example of images captured through crossed nicols observation.

FIG. 8 is a diagram schematically illustrating a configuration example of an endoscopic device that is an imaging device according to a second embodiment.

### MODE(S) FOR CARRYING OUT THE INVENTION

Hereinafter, embodiments of the present technology will be described with reference to the drawings.

### First Embodiment

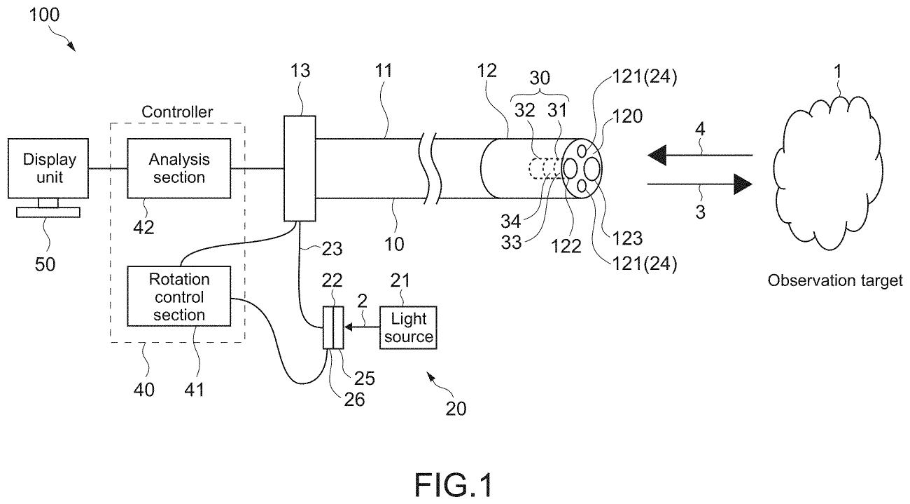

FIG. 1 is a diagram schematically illustrating a configuration example of an endoscopic device that is an imaging device according to a first embodiment of the present technology. An endoscopic device 100 includes an insertion unit 10, an illumination system 20, an imaging system 30, a controller 40, and a display unit 50. The endoscopic device 100 is capable of observing an observation target 1 such as a site of lesion by inserting the insertion unit 10 into a mouth or the like of a patient. In this embodiment, the observation target 1 is a body tissue.

The insertion unit 10 includes a soft section 11, a tip section 12, and an operation section 13. The soft section 11 has a soft tubular structure. The diameter, length, and the like of the soft section 11 are not limited, and may be appropriately set in accordance with the body shape of a patient, an insertion part of the patient such as a digestive tract or a trachea, or the like.

The tip section 12 is provided at one end of the soft section 11. The tip section 12 is inserted into the body of the patient, and is used for observation, treatment, or the like of the observation target 1. The tip section 12 includes a tip surface 120 that faces the observation target 1. The tip section 12 is bendable in a manner that the tip surface 120 faces various directions.

As illustrated in FIG. 1, the tip surface 120 has illumination openings 121, an imaging opening 122, and a treatment tool outlet 123. Through the treatment tool outlet 123, a treatment tool such as forceps or a snare moves in and out. The specific configuration of the tip surface 120 is not limited. For example, the tip surface 120 may be appropriately provided with a nozzle or the like that is an outlet of water, air, or the like.

The operation section 13 is provided with an operation handle for adjusting the direction of the tip surface 120, and various kinds of connectors such as a video connector or an optical connector (they are not illustrated by the drawings). In addition, the operation section 13 may be appropriately provided with a switch or the like that is necessary to operate the insertion unit 10.

The illumination system 20 includes a light source 21, a first polarization element 22, a polarization maintaining fiber 23, and an illumination lens 24. The light source 21 is installed separately from the insertion unit 10, and emits illumination light 2 toward the first polarization element 22. In this embodiment, non-polarized light is used as the illumination light 2. The non-polarized light does not have a specific polarization direction. As the light source 21, it is possible to use a white light emitting diode (LED), a high-pressure mercury lamp, or the like. Alternatively, any light source 21 capable of emitting non-polarized light can be used appropriately.

The first polarization element 22 polarizes at least part of illumination light 2 emitted from the light source 21, in a first polarization direction. In other words, the first polarization element 22 generates linearly polarized light of the first polarization direction, from the illumination light 2 incident on the first polarization element 22.

For example, in the case where the non-polarized illumination light 2 is incident on the first polarization element 22, the first polarization element 22 extracts a polarization component that vibrates in the first polarization direction, from the non-polarized illumination light 2. As described above, polarization of the illumination light 2 in the first polarization direction includes extraction of the polarization component of the first polarization direction from the non-polarized illumination light 2.

In this embodiment, an optical element (liquid crystal polarizer) is used as the first polarization element 22. The optical element includes a polarizing plate 25 and a liquid crystal variable wave plate 26. The polarizing plate 25 has a predetermined polarization axis, and is disposed fixedly with respect to the light source 21. The liquid crystal variable wave plate 26 is disposed across the polarizing plate 25 from the light source 21. Note that in FIG. 1, the polarization axis of the polarizing plate 25 is not illustrated for ease of explanation.

The polarizing plate 25 extracts linearly polarized light that vibrates in a direction parallel to the polarization axis of the polarizing plate 25, from the illumination light 2 incident on the polarizing plate 25. The polarization direction of the linearly polarized light that has been extracted is rotated by the liquid crystal variable wave plate 26, and then the linear polarization light is emitted. In other words, the linearly polarized light that has passed through the polarizing plate 25 and rotated by the liquid crystal variable wave plate 26 is the polarization light of the first polarization direction.

In addition, it is possible to arbitrarily set the first polarization direction by electrically controlling the liquid crystal variable wave plate 26. In other words, it is possible to generate linearly polarized light of any polarization direction by appropriately controlling a rotation angle of the linearly polarized light that has passed through the polarizing plate 25. In addition, when using the liquid crystal variable wave plate 26 rather than mechanically rotating the polarizing plate 25, it is possible to instantaneously change the first polarization direction, in other words, it is possible to quickly rotate the first polarization direction.

The specific configuration of the first polarization element 22 is not limited. For example, instead of the liquid crystal, it is possible to use an optical element using a transmissive ferroelectric substance such as PLZT. In addition, for example, an element capable of mechanically rotating the polarizing plate such as a wire grid polarizer or polarizing film may be used as the first polarization element 22. In addition, it is possible to appropriately configure the first polarization element 22 by using elements such as a polarizing plate or a wave plate.

The polarization maintaining fiber 23 is an optical fiber capable of transmitting light while substantially maintaining a polarization state of light. For example, the polarization maintaining fiber 23 is inserted into the operation section 13 from the first polarization element 22, passes through the inside of the soft section 11, and extends to the tip section 12. The polarization maintaining fiber 23 guides polarization light of the first polarization direction that has been emitted from the first polarization element 22, to the tip section 12 of the insertion unit 10 while substantially maintaining its polarization state. The specific configuration of the polarization maintaining fiber 23 is not limited. It is possible to appropriately use an optical fiber or the like capable of maintaining a polarization direction of linearly polarized light.

The illumination lenses 24 are disposed in the illumination openings 121 made in the tip surface 120 of the tip section 12. The illumination lens 24 magnifies the polarization light of the first polarization direction that has been passed through the polarization maintaining fiber 23, and emits the magnified light to the observation target 1. In FIG. 1, an arrow schematically represents polarization light 3 of the first polarization direction that is emitted from the illumination lenses 24. The specific configurations of the illumination lenses 24 are not limited. For example, any lenses capable of magnifying polarized illumination light may be used as the illumination lenses 24.

As described above, in the illumination system 20, the first polarization element 22 polarizes the illumination light 2 emitted from the light source 21 in the first polarization direction, and emits the polarized light to the observation target 1 via the polarization maintaining fiber 23 and the illumination lens 24. In this embodiment, the illumination system 20 corresponds to a first polarization section that irradiates a body tissue with polarization light polarized in the first polarization direction.

The imaging system 30 includes a second polarization element 31 and an image sensor 31, and is disposed inside the tip section 12. In FIG. 1, dotted lines schematically represents the imaging system 30 (the second polarization element 31 and the image sensor 32) disposed inside the tip section 12.

The second polarization element 31 is disposed in the imaging opening 122. Reflection light 4 is incident on the second polarization element 31. The reflection light 4 is the polarization light 3 reflected by the observation target 1. In FIG. 1, an arrow schematically represents the reflection light 4 reflected by the observation target 1. Note that sometimes the reflection light 4 may include polarization components in various polarization states.

Among beams of the reflection light 4 reflected by the observation target 1, the second polarization element 31 extracts a polarization component of a second polarization direction that intersects with the first polarization direction. In other words, the second polarization element 31 has a function of taking out the polarization component that vibrates in the second polarization direction from the reflection light 4 incident on the second polarization element 31.

In this embodiment, a liquid crystal polarizer including a liquid crystal variable wave plate 33 and a polarizing plate 34 is used as the second polarization element 31. As illustrated in FIG. 1, in the liquid crystal polarizer serving as the second polarization element 31, the liquid crystal variable wave plate 33 is disposed in a manner that the liquid crystal variable wave plate 33 faces the observation target 1, and the polarizing plate 34 is disposed on a side opposite to the side where the liquid crystal variable wave plate 33 faces the observation target 1.

The reflection light 4 is incident on the liquid crystal variable wave plate 33. The liquid crystal variable wave plate 33 rotates the entire reflection light 4 in a manner that a polarization component of the second polarization direction included in the reflection light 4 passes through the polarizing plate 34 in a subsequent stage.

For example, in the case where the second polarization direction is parallel to the polarization axis of the polarizing plate 34, the liquid crystal variable wave plate 33 transmits the reflection light 4 without rotating the reflection light 4. As a result, a polarization component that is included in the reflection light 4 and that is parallel to the polarization axis of the polarizing plate 34, that is, the polarization component of the second polarization direction passes through the polarizing plate 34, and is extracted. Alternatively, in the case where the second polarization direction is different from the polarization axis of the polarizing plate 34, the liquid crystal variable wave plate 33 rotates the entire polarization components included in the reflection light 4 in a manner that the second polarization direction becomes identical to the polarization axis of the polarizing plate 34 after the rotation. This makes it possible to extract the optical component of the second polarization direction.

In addition, it is possible to control the polarization component of the second polarization direction that is an extraction target, by controlling a rotation angle at the liquid crystal variable wave plate 33. For example, by appropriately setting the rotation angle at the liquid crystal variable wave plate 33, it is possible to extract a polarization component of a desired polarization direction (the second polarization direction) from the reflection light 4. It is also possible to quickly rotating the polarization direction (the second polarization direction).

The specific configuration of the second polarization element 31 is not limited. For example, instead of the liquid crystal, it is possible to use the optical element using the transmissive ferroelectric substance such as PLZT. In addition, for example, it is possible to use the element capable of mechanically rotating the wire grid polarizer, polarizing film, and the like. In addition, it is possible to appropriately configure the second polarization element 31 by using elements such as the polarizing plate and the wave plate. In this embodiment, the second polarization element 31 functions as a second polarization section.

The image sensor 32 is disposed across the second polarization element 31 from the observation target 1. In other words, the reflection light 4 is incident on the image sensor 32 from the observation target 1 via the second polarization element 31.

The image sensor 32 generates an image signal of the observation target 1 on the basis of the polarization component of the reflection light 4 extracted by the second polarization element 31. The image signal is a signal capable of constituting an image, and includes a plurality of pixel signals each including luminance information. The image consisting of the image signal may be a color image, a black and white image, or the like. In addition, for example, the luminance information includes information such as a luminance value of each pixel, and RGB values indicating intensities of respective colors including red R, green G, and blue B of each pixel. The type, format, and the like of the image signal are not limited. Any format of the image signal may be used. The generated image signal is output to the controller 40. In this embodiment, the image sensor 32 corresponds to a generation section.

As the image sensor 32, it is possible to use a charge coupled device (CCD) sensor, a complementary metal oxide semiconductor (CMOS) sensor, or the like, for example. Of course, it is possible to use another type of sensor.

The controller 40 includes hardware that is necessary for configuring a computer such as a CPU, ROM, RAM, and an HDD. An imaging method according to the present technology is executed when the CPU loads a program into the RAM and executes the program. The program is recorded in the ROM or the like in advance. For example, the controller 40 can be implemented by any computer such as a personal computer (PC).

As illustrated in FIG. 1, in this embodiment, a rotation control section 41 and an analysis section 42 are configured as functional blocks when the CPU executes a predetermined program. Of course, it is also possible to use dedicated hardware such as an integrated circuit (IC) to implement each of the blocks. The program is installed in the controller 40 via various kinds of recording media, for example. Alternatively, it is also possible to install the program via the Internet.

The rotation control section 41 is capable of rotating each of the first polarization direction and the second polarization direction. For example, the rotation control section 41 outputs respective control signals or the like to the first polarization element 22 and the second polarization element 31 for setting angles of the first and second polarization directions. This makes it possible to appropriately rotate each of the first polarization direction and the second polarization direction.

For example, by rotating the first polarization direction, it is possible to control the polarization direction of the polarization light to be emitted to the observation target 1. In addition, for example, it is possible to control the polarization direction of the polarization component extracted from the reflection light 4 by rotating the second polarization direction.

The rotation control section 41 rotates each of the first polarization direction and the second polarization direction while maintaining an intersection angle between the first polarization direction and the second polarization direction. For example, the rotation control section 41 outputs respective control signals that instruct the first polarization element 22 and the second polarization element 31 to rotate the first polarization direction and the section polarization direction by predetermined angles. This makes it possible to perform rotation operation for rotating the first polarization direction and the second polarization direction by the predetermined angle while maintaining the intersection angle between the first polarization direction and the second polarization direction.

In addition, the rotation control section 41 rotates the first polarization direction and the second polarization direction in synchronization with each other. For example, the rotation control section 41 generates a synchronization signal such as a clock signal, and controls the first polarization element 22 and the second polarization element 31 in synchronization with each other on the basis of the synchronization signal. This makes it possible to rotate the first and second polarization directions at substantially the same timings. Note that the rotation control section 41 is capable of outputting the synchronization signal to the image sensor 32 or the like.

The analysis section 42 analyzes the image signal of the observation target 1 generated by the image sensor 32. In addition, the analysis section 42 generates an intraoperative image of the observation target 1 on the basis of an analysis result of the image signal. The intraoperative image is an image of the observation target 1 captured during a surgery including observation, treatment, and the like performed by using the endoscopic device 100. Details of operation and the like of the analysis section 42 will be described later.

The display unit 50 displays the intraoperative image of the observation target 1 generated by the analysis section 42. For example, a display device such as a liquid crystal monitor is used as the display unit 50. For example, the display unit 50 is installed in a room where endoscopic observation is performed. This makes it possible for a doctor to perform observation and treatment while watching the intraoperative image displayed on the display unit 50. The specific configuration of the display unit 50 is not limited. For example, as the display unit 50, it is possible to use a head-mounted display (HMD) or the like capable of displaying the intraoperative image.

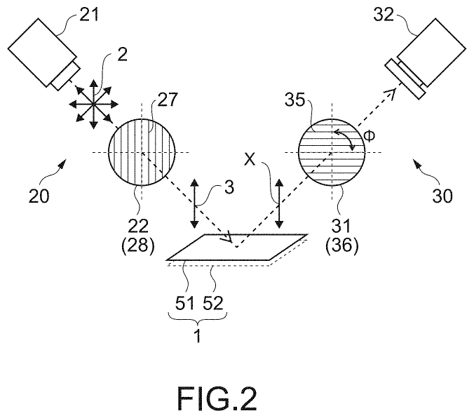

FIG. 2 is a schematic diagram illustrating an example of reflection by the observation target 1. With reference to FIG. 2, reflection by a surface 51 of the observation target 1 will be described. FIG. 2 schematically illustrates the light source 21 and the first polarization element 22 as the illumination system 20. Illustration of the polarization maintaining fiber 23 and the illumination lens 24 described with reference to FIG. 1 is omitted. In addition, as the imaging system 30, FIG. 2 schematically illustrates the second polarization element 31 and the image sensor 32.

To simplify the explanation, in FIG. 2, a polarizing plate 28 having a first polarization axis 27 represents the first polarization element 22 including the polarizing plate 25 and the liquid crystal variable wave plate 26. Among beams of the illumination light 2, the first polarization element 22 emits a polarization component of a direction parallel to the first polarization axis 27 as the polarization light 3 of the first polarization direction. This corresponds to a case where the liquid crystal variable wave plate 26 rotates the polarization direction of linearly polarized light extracted by the polarizing plate 25, and the linearly polarized light is emitted as the polarization light 3 of the first polarization direction.

In addition, a polarizing plate 36 having a second polarization axis 35 represents the second polarization element 31 including the polarizing plate 34 and the liquid crystal variable wave plate 33. The second polarization element 31 extracts a polarization component parallel to the second polarization axis 35 as a polarization component of the second polarization direction. This corresponds to a case where the liquid crystal variable wave plate 33 rotates reflection light 4 in a manner that the polarization component of the second polarization direction passes through the polarizing plate 34.

The first and second polarization directions are rotated by electrically controlling the liquid crystal variable wave plates 26 and 33 when the polarizing plates 28 and 36 illustrated in FIG. 2 are rotated. Note that the polarizing plates 28 and 36 are installed as the structural elements that are schematically illustrated in FIG. 2, that is, the first polarization element 22 and the second polarization element 31. In addition, mechanisms for physically rotating them are also included in the configurations of the first and second polarization sections according to the present technology.

In the example illustrated in FIG. 2, an intersection angle Φ between the first and second polarization directions is set to approximately 90 degrees, and the first and second polarization directions establish a substantially crossed nicols relation.

As illustrated in FIG. 2, in the illumination system 20, the light source 21 emits the non-polarized illumination light 2. Among beams of the illumination light 2, the first polarization element 22 extracts a polarization component of a direction parallel to the first polarization axis 27 as the polarization light 3 of the first polarization direction. The extracted polarization light 3 is emitted toward the observation target 1.

Part of the polarization light 3 incident on the observation target 1 is reflected near the surface 51 of the observation target 1. Most of the reflection near the surface 51 of the observation target 1 is surface reflection. With regard to the surface reflection, a polarization state of light reflected by the reflection surface hardly changes from a polarization state of light incident on a reflection surface (the surface 51 of the observation target 1). This means that, the polarization state is maintained before and after the reflection.

Therefore, as illustrated in FIG. 2, reflection light 4a reflected near the surface 51 of the observation target 1 proceeds to the imaging system as linearly polarized light that maintains the first polarization direction but is affected by properties of the vicinity of the surface of the observation target. Note that the other portions of the polarization light 3 incident on the observation target 1 are diffused/scattered through an inside 52 of the observation target 1 and reflected while their polarization directions are randomized due to multiple reflection.

The reflection light 4a polarized in the first polarization direction is incident on the second polarization element 31 of the imaging system 30. Since the first and second polarization directions establish the substantially crossed nicols relation, a polarization plane of the reflection light 4a polarized in the first polarization direction is kept by the surface reflection. Therefore, the reflection light 4a hardly passes through the second polarization element 31, and most of the reflection light 4a are absorbed/reflected by the second polarization element 31. As a result, the reflection light 4a reflected near the surface 51 of the observation target 1 is hardly received by the image sensor 32 in the subsequent stage after the second polarization element 31.

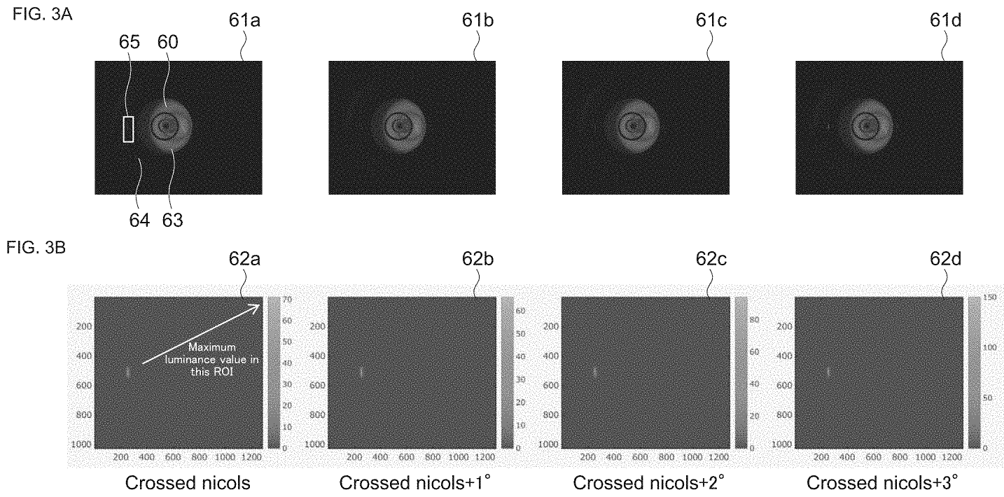

FIG. 3 is a diagram illustrating specific examples of specular reflection. FIG. 3A illustrates images 61a to 61d of a level 60 captured via the second polarization element 31 in the cases where an intersection angle Φ of the first and second polarization directions Φ is 90°, 91°, 92°, and 93°. FIG. 3B illustrates maps 62a to 62d showing reflection light intensity distributions with regard to the images 61a to 61d.

The level 60 includes a cylindrical bubble tube 63 at its center, and includes a metal frame 64 around the cylindrical bubble tube 63. The images 61a to 61d of the level 60 show images of the level 60 by reflection light diffusely reflected by the cylindrical bubble tube 63 and reflection light specularly reflected by the metal frame 64. Each of the images has been captured in a near-crossed nicols state. Therefore, the reflection light specularly reflected by the metal surface of the metal frame 64 is hardly received, and the metal frame 64 is displayed darkly.

The maps 62a to 62d illustrated in FIG. 3B show luminance distributions of gray scale luminance values in an analysis region (region of interest (ROI) 65). The ROI 65 is displayed in the image 61a. A vertical axis and a horizontal axis of each map correspond to the number of vertical and horizontal pixels in each image of the level. Gray scale bars represent luminance values in the ROI 65. The ROI 65 is set in a manner that the ROI 65 is disposed on a boundary between the cylindrical bubble tube 63 and the metal frame 64.

In ideal crossed nicols observation, a specular reflection component is zero. In practice, some specular reflection components remain because of attenuation (extinction ratio) of polarization components parallel to the polarization axis of the polarizing plate, wavelength dependency of the polarizing plate, an incident angle on a subject (the observation target 1), deviation from an orthogonal state, or the like. For example, in the map 62a of a crossed nicols state where the intersection angle Φ is 90°, a slight specular reflection component remains in the ROI. With regard to the map 62a, the maximum luminance value in the ROI 65 is 71.

In the case where the intersection angle Φ between the first and second polarization directions deviates from the crossed nicols state (Φ=90°) by 1° (the map 62b), the maximum luminance value in the ROI 65 is 66. In a similar way, in the case where the intersection angle Φ deviates by 2° (the map 62c), the maximum luminance value in the ROI 65 is 94. In the case where the intersection angle Φ deviates by 3° (the map 62d), the maximum luminance value in the ROI 65 is 150. Note that the maximum luminance values of the respective maps correspond to maximum values (brightest values) of the respective gray scale bars.

As described above, when the intersection angle Φ between the first and second polarization directions deviates from the crossed nicols state by 3° more, the number of specular reflection components included in the reflection light 4a is suddenly increased. For example, the specular reflection components may be a cause of halation, reflected glare of illumination light (polarization light 3), or the like when the observation target 1 is observed. In addition, there is a possibility that the specular reflection component causes noise at a time of crossed nicols observation. Therefore, in the case where the intersection angle Φ deviates from the crossed nicols state by 3° or more, there is a possibility that effects of the reflected glare of illumination light or the like increases.

In this embodiment, the intersection angle Φ between the first and second polarization directions is set to an angle in a range of 90°±2°. When the intersection angle Φ is set to the range of 90°±2°, it is possible to sufficiently attenuate the specular reflection component, and it is possible to sufficiently attenuate the reflected glare of illumination light. A surface reflection component of a body tissue is considered to be smaller than the specular reflection component of the metal material. Therefore, it is possible to accurately observe the observation target 1, and this makes it possible to sufficiently support observation of the body tissue.

Note that the range of the intersection angle Φ between the first and second polarization directions is not limited. The range of the intersection angle Φ may be appropriately set in a range capable of achieving acceptable observation accuracy. For example, the intersection angle Φ may be set to an angle in a range wider than 90°±2° such as 90°±5° or 90°±10°. For example, it is possible to appropriately set the intersection angle Φ in accordance with the type of observation target 1 and characteristics of the illumination system 20 and the imaging system 30.

A method of setting the intersection angle Φ between the first and second polarization directions to a desired value such as 90°±2° is not limited. For example, the intersection angle Φ may be set on the basis of a polarization component of the first polarization direction included in the reflection light 4a, that is, the specular reflection component.

For example, in FIG. 2, a sample including a metal surface with strong specular reflectivity is used as the observation target 1. First, the first polarization axis 27 of the first polarization element 22 is fixed, and illumination light (the polarization light 3) is emitted to the metal surface. The reflection light 4a polarized in the first polarization direction is emitted from the metal surface, and is incident on the second polarization element. Here, the second polarization axis 35 of the second polarization element 31 is rotated, and a total amount of light received by the image sensor 32 is detected.

For example, in the case where the first polarization direction is parallel to the second polarization axis 35, the reflection light 4a polarized in the first polarization direction substantially passes through the second polarization element 31, and the total amount of light received by the image sensor 32 becomes maximum. Accordingly, it is possible to set the intersection angle Φ between the first and second polarization direction to 90° by rotating the second polarization axis 35 by 90° on the basis of the angle at which the total amount of light is maximum. Of course, it is also possible to set the intersection angle Φ on the basis of an angle at which the total amount of light is minimum. In addition, it is possible to use any method capable of setting the intersection angle Φ.

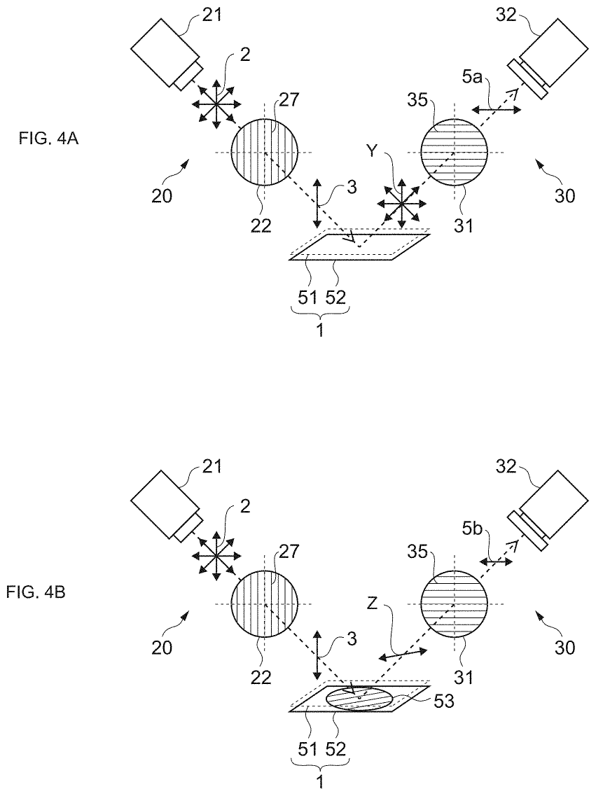

FIG. 4 is a schematic diagram illustrating an example of reflection caused in the inside 52 of the observation target 1. In FIG. 4A to FIG. 4C, the first polarization element 22 and the second polarization element 31 are disposed so as to establish the substantially crossed nicols relation.

As illustrated in FIG. 4, the polarization light 3 of the first polarization direction emitted from the illumination system 20 is incident on the observation target 1. Part of the polarization light 3 incident on the observation target 1 is specularly reflected by the surface 51 of the observation target 1, and the other portions of the polarization light 3 are incident on the inside 52 of the observation target 1.

The inside 52 of the observation target 1 includes various kinds of body tissues such as fat and muscle. The polarization light 3 is diffused or scattered, or a polarization direction of the polarization light 3 is rotated in accordance with optical characteristics of respective body tissues. As a result, as illustrated in FIG. 4A, reflection light 4b multiply scattered in the inside 52 of the observation target 1 includes polarization components of various polarization directions.

The reflection light 4b reflected in the inside 52 of the observation target 1 is incident on the second polarization element 31. The second polarization element 31 extracts a polarization component of the reflection light 4b parallel to the second polarization axis 35 as a polarization component 5a of the second polarization direction. The extracted polarization component 5a is incident on the image sensor 32.

FIG. 4B is a schematic direction illustrating a case where the polarization light 3 of the first polarization direction is incident on an anisotropic body 53 in the inside 52 of the observation target 1. Here, for example, the anisotropic body 53 is an optically anisotropic body tissue. Examples of the anisotropic body 53 of the body tissue include muscle fibers of muscle, collagen fibers in cartilage such as a meniscus, and a nerve fascicles that are bundles of nerve fibers. Of course, the present technology is not limited thereto. The present technology is applicable to any optically anisotropic tissue and the like.

For example, when the linearly polarized light is emitted to the anisotropic body 53, the polarization state changes in accordance with the optical characteristics of the anisotropic body 53. For example, due to optical rotation of the anisotropic body 53, a polarization direction of the linearly polarized light is rotated. In addition, due to circular dichroism of the anisotropic body 53, some polarization components of the linearly polarized light are absorbed and the linearly polarized light is polarized as elliptically polarized light. As a result, the anisotropic body 53 emits reflection light 4c in the polarization state different from that of the linearly polarized light emitted to the anisotropic body 53.

In addition, the polarization states of the reflection light 4c such as the polarization direction and ellipticity are changed in accordance with the polarization direction of the linearly polarized light that has been emitted. In other words, the polarization state, intensity, and the like of the reflection light 4c are changed in accordance with optical characteristics of the anisotropic body 53 and the polarization direction of the linearly polarized light emitted to the anisotropic body 53.

As illustrated in FIG. 4B, the polarization light 3 of the first polarization direction is emitted to the anisotropic body 53. The anisotropic body 53 emits the reflection light 4c whose polarization state has been changed. Note that FIG. 4B schematically illustrates the reflection light 4c as the linearly polarized light. However, the present technology is not limited thereto. Sometimes elliptically polarized light or the like may be emitted as the reflection light 4c.

The reflection light 4c reflected by the anisotropic body 53 is incident on the second polarization element 31. The second polarization element 31 extracts a polarization component 5b of the second polarization direction among polarization components included in the reflection light 4c. The extracted polarization component 5b is emitted toward the image sensor 32.

When extracting the polarization component 5b, the second polarization element 31 reflects/absorbs a polarization component of the reflection light 4c that is orthogonal to the second polarization direction. Therefore, intensity (amount of light) of the extracted polarization component 5b varies depending on the polarization state of the reflection light 4c polarized by the anisotropic body 53. Note that in FIG. 2, the intensity of the polarization component 5b is indicated by a length of an arrow representing the polarization component 5b.

Here, it is assumed that the first and second polarization directions are rotated while maintaining the crossed nicols relation. In this case, a polarization direction (the first polarization direction) of linearly polarized light emitted to the anisotropic body 53, and a polarization direction (the second polarization direction) of the polarization component 5b extracted by the second polarization element 31 are changed. Therefore, intensity of the polarization component 5b extracted by the second polarization element 31 is changed. As described above, in the crossed nicols observation, the intensity of transmitted light (the polarization component 5b) that has passed through the second polarization element 31 is changed with rotation of the first and second polarization directions.

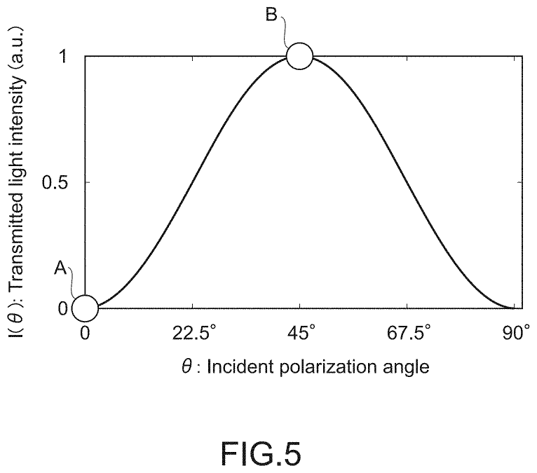

FIG. 5 is a graph showing intensity of transmitted light that has passed through the second polarization element 31 in the case of performing crossed nicols observation of the anisotropic body 53. A horizontal axis of the graph represents incident polarization angles θ, and a vertical axis represents intensities I of transmitted light that has passed through the second polarization element 31.

The incident polarization angle θ is an angle of a polarization direction of linearly polarized light with respect to the anisotropic body 53 in the case where the anisotropic body 53 is irradiated with the linearly polarized light. The incident polarization angle θ is set on the basis of the minimum transmitted light intensity I, for example. In other words, the incident polarization angle θ is set in a manner that the incident polarization angle θ (an angle of a polarization direction of linearly polarized light) is 0° with respect to the anisotropic body 53 in the case of the minimum transmitted light intensity I. Note that a method or the like of setting the incident polarization angle θ is not limited.

In the case where the anisotropic body 53 is observed in a state where the first and second polarization directions establish the crossed nicols relation, the transmitted light intensity I is expressed as the following equation using the incident polarization angle θ.

I(θ)=I₀·sin²(2θ)·sin²(δ/2)

where I₀ represents intensity of a transmitted light that has passed through the second polarization element 31 in a state of a parallel nicols relation in which the first polarization direction is parallel to the second polarization direction. In addition, δ represents a phase difference caused by the anisotropic body 53. δ is a value depending on optical characteristics or the like of the anisotropic body 53.

As indicated in the above-listed equation, the transmitted light intensity I(θ) is a periodic function with a period of 90° with regard to the incident polarization angle θ. FIG. 5 illustrates the graph of transmitted light intensities I(θ) of the incident polarization angles θ of 0° to 90° on the basis of the incident polarization angle θ of 0° at which the minimum transmitted light intensity I(θ) is obtained.

As illustrated in FIG. 5, in the case where the incident polarization angle θ is 0° (a data point A in the graph), the transmitted light intensity I(θ) is zero, which is the minimum value. Note that sometimes the minimum value of the transmitted light intensity I(θ) is not zero because a certain type of the anisotropic body 53 to be observed causes random polarization due to its internal multiple reflection.

The value of the transmitted light intensity I(θ) increases as the incident polarization angle θ gets larger than the data point A. Subsequently, the transmitted light intensity I(θ) becomes maximum when the incident polarization angle θ becomes 45° (a data point B in the graph). FIG. 5 illustrates a normalized graph in which the maximum value of the transmitted light intensity I(θ) is 1. If the graph is not normalized, for example, the maximum value of the transmitted light intensity I(θ) is a value depending on the type or the like of the anisotropic body 53.

As the incident polarization angle θ gets larger than the data point B, the transmitted light intensity I(θ) decreases. When θ=90°, the transmitted light intensity I(θ) reaches zero. When the incident polarization angle θ changes as described above, the transmitted light intensity (θ) becomes the minimum value (or the maximum value) with a period of 90°. Therefore, for example, when the incident polarization angle θ is rotated by 360°, the transmitted light intensity I(θ) becomes minimum (or maximum) four times.

As illustrated in FIG. 5, in the crossed nicols observation, an intensity difference in the transmitted light intensity I(θ) between the incident polarization angles θ of 0° and 45° is maximum. In other words, change in the transmitted light intensity I(θ) of a certain anisotropic body 53 becomes maximum when the incident polarization angle θ is changed from 0° to 45°.

For example, it is assumed that the crossed nicols observation is performed on the observation target 1 including an anisotropic body 53 of interest. In this case, for example, an image of the observation target 1 captured at θ=0° is compared with an image of the observation target 1 captured at θ =45°. This makes it possible to detect largest change in brightness (luminance difference) of a region including the anisotropic body 53. Accordingly, it is possible to accurately detect the anisotropic body 53 included in the observation target 1.

In the endoscopic device 100, the incident polarization angle θ is changed by rotating the first and second polarization directions. For example, states of the first and second polarization directions before rotation are referred to as a first state. In addition, states of the first and second polarization directions after rotation by a rotation angle ω is referred to as a second state. In this embodiment, the first state corresponds to a predetermined state, and the rotation angle ω corresponds to a predetermined angle.

For example, a state where the minimum transmitted light intensity I(θ) is obtained is set as the first state, and the rotation angle ω is set to 45°. This makes it possible to achieve the first state corresponding to θ=0°, and the second state corresponding to θ=45°. Accordingly, it is possible to accurately detect the anisotropic body 53 included in the observation target 1.

In the case where the first and second polarization directions deviate from the crossed nicols relation, the intensity difference between θ=0° and θ=45° is smaller than the case where the crossed nicols relation is established. In other words, in the state where the first and second polarization directions deviate from the crossed nicols relation, a difference between the maximum value and the minimum value in the graph of the transmitted light intensity I(θ) gets smaller. Accordingly, in the case of observing the anisotropic body 53, it is desirable to set the intersection angle Φ between the first and second polarization directions to 90°, and set the incident polarization angles θ to 0° and 45°. This makes it possible to maximize the intensity difference in the transmitted light intensity I(θ).

Note that a sufficiently large intensity difference in the transmitted light intensity I(θ) is obtained at the incident polarization angles θ of 0° and 45° even if the intersection angle Φ between the first and second polarization directions slightly deviates from 90°. For example, in the range (90°±2°) of the intersection angle Φ described with reference to FIG. 3 and the like, it is possible to sufficiently suppress a noise component due to specular reflection or the like on the observation target 1. Even in the case where the intersection angle Φ slightly deviates from such a crossed nicols state by ±2°, it is possible to detect the anisotropic body 53 or the like with sufficient accuracy. Of course, it is also possible to set the intersection angle Φ to an angle in a wider range than 90°±2°.

The rotation angle ω of rotating the first and second polarization directions while maintaining the crossed nicols (substantially crossed nicols) relation is not limited to 45°. As illustrated in FIG. 5, for example, an intensity difference between θ=0° and θ=22.5° is approximately 50% (approximately −3 dB) of an intensity difference between θ=0° and θ=45°. In a similar way, an intensity difference between θ=0° and θ=67.5° is approximately 50% of an intensity difference between θ=0° and θ=45°. Even in this case, it is also possible to sufficiently detect change in brightness of the region including the anisotropic body 53.

In this embodiment, the rotation angles co of the first and second polarization directions are set to angles in a range of 45°±22.5°. In other words, each of the first polarization direction and the second polarization direction is rotated within a range equal to or more than 22.5° and equal to or less than 67.5°. This makes it possible to accurately detect the anisotropic body 53. Of course, the present technology is not limited to this range. Any other range may be set.

For example, in the case where an observation environment includes large amounts of noise, the range of the rotation angle ω may be set to a narrow range such as 45°±1°. Alternatively, in the case where the noise is sufficiently small, it is possible to set the range of the rotation angle ω to a range wider than 45°±22.5°. This makes it possible to increase a degree of freedom of setting the rotation angle co. Alternatively, it is possible to appropriately set the range of rotation angle ω in accordance with the type of anisotropic body 53, characteristics of the illumination system 20 and the imaging system 30, and the like.

Note that sometimes a body tissue includes a plurality of anisotropic bodies 53 having different characteristics from each other. For example, the incident polarization angle θ is set on the basis of a transmitted light intensity I(θ) of one of the anisotropic bodies 53. Subsequently, each of the first and second polarization directions is rotated in a manner that the incident polarization angle θ is changed from 0° to 45° while maintaining the crossed nicols state with respect to the anisotropic body 53 for which the incident polarization angle θ is set.

In this case, typically, the rotation does not change incident polarization angles θ of the other anisotropic bodies 53 from 0° to 45°. However, luminance differences corresponding to respective optical characteristics are detected. Therefore, in addition to detection of a single anisotropic body 53, it is also possible to detect the plurality of other anisotropic bodies 53 distinctively from each other by analyzing intensity differences in transmitted light among the plurality of anisotropic bodies 53. This makes it possible to detect a plurality of tissues having different optical anisotropies.

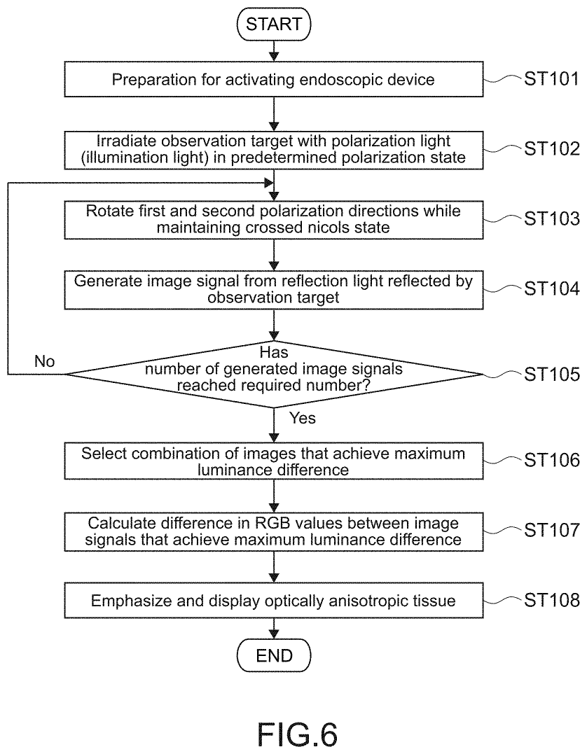

FIG. 6 is a flowchart illustrating an example of observation of a body tissue. Preparation for activating the endoscopic device 100 is performed (Step 101). For example, respective sections such as the light source 21, the image sensor 32, and the controller 40 are activated. In addition, an operator such as a doctor inputs various kinds of parameters for observation using the endoscopic device 100 (such as amounts of light of the light source 21 and sensitivity of the image sensor 32) to the controller 40 or the like.

Polarization light in a predetermined polarization state is generated from the illumination light 2, and the polarization light is emitted to the observation target 1 (Step 102). In other words, the first polarization element 22 generates the polarization light 3 of the first polarization direction (polarization light in the predetermined polarization state), and the polarization light 3 is emitted to the observation target 1. The first polarization direction is set to a default direction (angle) that has been set in advance. Alternatively, the first polarization direction may be set to an angle desired by the operator. Alternatively, it is possible to automatically set the first polarization direction on the basis of information of optical characteristics of the observation target 1. For example, an angle at which the minimum transmitted light intensity I(θ) is obtained is estimated, and the first polarization direction may be set to this angle.

The second polarization direction is set in a manner that the substantially crossed nicols relation is established between the first polarization direction and the second polarization direction.

The rotation control section 41 rotates the first and second polarization directions while maintaining the substantially crossed nicols state (Step 103). In this embodiment, each of the polarization directions is rotated by an imaging angle d that has been set in advance. The imaging angle d is a value different from the rotation angle ω for changing the first state to the second state. Therefore, a state before rotation by the imaging angle d and a state after the rotation by the imaging angle d do not correspond to the first state and the second state. Details thereof will be described later.

in addition, the rotation may be omitted in the case where the process in Step 103 is performed for the first time after the preparation for activation is performed in Step 101. In other words, when the process in Step 103 is performed for the first time, rotation is made by an imaging angle d=0°. When the process in Step 103 is performed for the second or subsequent times, the first and second polarization direction are rotated by an imaging angle d.

On the basis of the reflection light 4 reflected by the observation target 1, the image sensor 32 generates an image signal of the observation target 1 (Step 104). In other words, the image signal is generated on the basis of transmitted light (polarization components 5a and 5b) that has passed through the second polarization element 31 among beams of the reflection light 4 reflected by the observation target 1. In this embodiment, it is possible to generate the image signal capable of configuring a color image of the observation target 1. Of course, it is also possible to generate an image signal capable of configuring a black and white image or the like. The generated image signal is output to the analysis section 42.

It is determined whether or not the number of generated image signals has reached a required number (Step 105). In the case where it is determined that the number of image signals has not reached the required number (No in Step 105), the process returns to Step 103 and a loop process is executed.

Next, the imaging angle d used in Step 103 and the required number used in Step 105 will be described. As described with reference to FIG. 5, a difference in the incident polarization angle θ between the maximum transmitted light intensity I(θ) of the anisotropic body 53 and the minimum transmitted light intensity I(θ) of the anisotropic body 53 is 45°. Therefore, in this embodiment, the rotation angle ω for changing the first state to the second state is set to 45°.

The imaging angle d used in Step 103 is set on the basis of the rotation angle ω of 45°. For example, the imaging angle d is set to an angle obtained by dividing the rotation angle ω of 45°. The imaging angle d is represented as d=45°/n, where n is an integer equal to or more than 1. Rotation by the rotation angle ω is made when each of the first and second polarization directions is rotated by the imaging angle d for n-th times. Accordingly, a state before rotating the first and second polarization directions by the imaging angle d for n-th times corresponds to the first state, and a state after rotating the first and second polarization directions by the imaging angle d for n-th times corresponds to the second state.

For example, the required number used in Step 105 is set to a number that satisfies a condition that an image is captured at a rotation position close to an incident polarization angle at which the minimum transmitted light intensity I(θ) is obtained as illustrated in FIG. 5. As described with reference to FIG. 5, an angle at which the minimum transmitted light intensity I(θ) is obtained has a period of 90°. Therefore, the first polarization direction is repeatedly rotated by the imaging angle d until a range equal to or more than 90° is obtained. Accordingly, at least one of a plurality of rotation positions becomes a rotation position close to an incident polarization angle at which the minimum transmitted light intensity I(θ) is obtained.

For example, a minimum value of m is set in a manner that the expression “Imaging angle d×Integer m≥90°” is satisfied. In other words, the minimum number (m) of rotations (repetitions) is set in a manner that an angle equal to or more than 90° is obtained when rotation by an imaging angle d is repeated. In this embodiment, the imaging angle d=45°/n. Therefore, the required number used in Step 105 is m=2n.

When any direction is 0°, a range from 0° to md)(=90° includes an incident polarization angles at which the minimum transmitted light intensity I(θ) is obtained. In addition, a transmitted light intensity I(θ) at 0° is equal to a transmitted light intensity I(θ) at md)(=90°. Accordingly, at least one of m number of rotation positions including 0°, d, 2d . . . (m−1)d becomes a rotation position close to an angle at which the minimum transmitted light intensity I(θ) is obtained.

Therefore, when m is set to the required number used in Step 105, it is possible to satisfy the condition that an image is captured at the rotation position close to the incident polarization angle at which the minimum transmitted light intensity I(θ) is obtained as illustrated in FIG. 5. Of course, a method of setting the required number used in Step 105 is not limited to the setting method that complies with the above-described condition.

Here, a case where the imaging angle d used in Step 103 is set to d=45°/2=22.5° will be described. In this case, 4d=4×22.5°=90°. Therefore, the required number of repetitions of Step 105 is four.

The first and second polarization directions are rotated by an imaging angle d=22.5° several times in a manner that a rotation position of 0°, 22.5°, 45°, and 67.5° are obtained with respect to the initial position set in Step 102. One of the four rotation positions is a rotation position close to the angle at which the minimum transmitted light intensity I(θ) is obtained.

In the case where differences between the respective rotation positions of 0° to 67.5° and the angle at which the minimum transmitted light intensity I(θ) is obtained become maximum, the angle at which the minimum transmitted light intensity I(θ) is obtained is located at midpoints of the respective rotation positions of 0° to 67.5°. Therefore, at a maximum, the difference is 11.25°, which is a half value of the imaging angle d=22.5°. In other words, at a maximum, the difference between the angle of image capturing and the angle at which the minimum transmitted light intensity I(θ) is obtained is the half value of the imaging angle d.

For example, as illustrated in FIG. 5, a transmitted light intensity I(θ) obtained in the case where the incident polarization angle θ of the first polarization direction is 11.25° is compared with a transmitted light intensity I(θ) obtained in the case where the incident polarization angle θ is rotated by 45° and becomes 56.25°. In this case, the intensity difference is approximately 70% of an intensity difference between an incident polarization angle θ of 0° and an incident polarization angle θ of 45°. Accordingly, it is possible to obtain the sufficiently large value. Therefore, it is possible to accurately detect the anisotropic body 53 by setting the imaging angle d to 22.5° and capturing an image for times while maintaining the crossed nicols state.

It is also possible to set the imaging angle d on the basis of a difference from the angle at which the minimum transmitted light intensity I(θ) is obtained. For example, the imaging angle d may be set to a value obtained by doubling an acceptable angular difference (in this example, 11.25°).

Note that in the case where the imaging angle d is set to a different value from d=45°/n and an imaging angle d×m=90° is not satisfied, it is sufficient to make rotation a number of times obtained by adding 1 to the minimum value of m that satisfies a condition that the imaging angle d×m>90°. The range of 0° to dm)(>90° includes the angle at which the minimum transmitted light intensity I(θ) is obtained. Therefore, one of (m+1) number of rotation positions is a rotation position close to the angle at which the minimum transmitted light intensity I(θ) is obtained.

It is assumed that the required number of image signals are obtained in Step 105 (Yes in Step 105). In other words, respective image signals are obtained in the case where rotation is made by the imaging angle d=22.5° four times. This means that a combination of image signals are generated in the case where the first and second polarization direction are rotated by the rotation angle ω=45°.

In other words, the image sensor 32 generates first image signals in the case where each of the first and second polarization directions is in the first state before the rotation. In addition, the image sensor 32 generates second image signals in the case where the rotation control section 41 rotates each of the first and second polarization directions by the rotation angle ω=45° in the first state.

For example, in the case where the rotation positions are a combination of (0°, 45°), an image obtained at a rotation position of 0° corresponds to the first image signal, and an image obtained at a rotation position of 45° corresponds to the second image signal. In the case where the rotation positions are a combination of (22.5°, 67.5°), an image obtained at a rotation position of 22.5° corresponds to the first image signal, and an image obtained at a rotation position of 67.5° corresponds to the second image signal.

The analysis section 42 selects a combination of image signals that achieve the maximum luminance difference (Step 106). In this embodiment, as the combination of images that achieve the maximum luminance difference, it is possible to select combinations of a first image signal and a second image signal in which incident polarization angles θ are different from each other by 45°. In other words, a pair of (0°, 45°) and a pair of (22.5°, 67.5°) are selected. Of course, instead of or in addition to the information of rotation positions, it is possible to select a combination of images that achieve the maximum luminance difference on the basis of luminance information included in image signals obtained at the respective rotation positions.

The analysis section 42 calculates a difference in RGB values between the image signals that achieve the maximum luminance difference (Step 107). In this embodiment, the process of calculating differences in the RGB values is performed on the respective combinations of the first image signal and the second image signal in which incident polarization angles θ are different from each other by 45°.

The first image signal includes a plurality of first pixel signals each including luminance information such as RGB values. In addition, the second image signal includes a plurality of second pixel signals each including luminance information such as RGB values.

The analysis section 42 calculates the differences in RGB values on the basis of RGB values of the first pixel signals and the second pixel signals corresponding to each other. By calculating the differences between the RGB values, it is possible to calculate how brightness of each pixel has changed, as color information.

In addition, the analysis section 42 calculates luminance differences between the first pixel signals and the second pixels signals from the differences in the RGB values. For example, it is assumed that (dR, dG, dB) is calculated as a difference in RGB values of a certain pixel. In this case, for example, a luminance difference dY is calculated by using the following equation of converting the RGB values into a luminance value Y.

dY=0.299dR+0.587dG+0.114dB

By calculating differences in the RGB values of respective pixels as described above, it is possible to calculate differences in brightness (luminance differences) of respective pixels. A method or the like of calculating the luminance difference is not limited. For example, a sum of differences in RGB values of respective pixels may be calculated as the luminance difference. Alternatively, it is possible to use any method of calculating luminance differences.

The process of calculating luminance differences is performed on each combination of the first and second image signals. The analysis section 42 detects a combination including pixels with the maximum luminance difference. This makes it possible to select a combination having the maximum change in the transmitted light intensity I(θ) of the anisotropic body 53. In other words, a combination of image signals captured at the incident polarization angles θ of approximately 0° and 45° is selected.

As described above, in Step 106, differences in RGB values between the first image signal and the second image signal are calculated. With regard to the first and second image signals, the incident polarization angles θ are different from each other by 45°. In addition, on the basis of the calculated differences, the image signals captured at the incident polarization angles θ of approximately 0° and 45° are specified. Accordingly, it is possible to accurately detect the anisotropic body 53 included in the observation target 1.

Note that a method of detecting the combinations is not limited. For example, among the image signals, it is possible to detect a combination on the basis of luminance values of pixels included in a predetermined region. In other words, it is possible to focus on the predetermined region of the observation target 1 and select a combination of image signals on the basis of change in brightness of respective pixels within the region. This makes it possible to accurately observe an anisotropic body 53 of the focused part.

The analysis section 42 generates an image in which an optically anisotropic tissue is emphasized, and the display unit 50 displays the generated image (step 108). In this embodiment, the image to be displayed on the display unit 50 is generated on the basis of the first and second image signals with the maximum luminance difference detected in Step 106.

The analysis section 42 extracts a part having a luminance difference higher than a predetermined threshold. For example, the predetermined threshold is set on the basis of a background noise level or the like of the imaging system 30. This makes it possible to remove noise other than change in brightness caused by rotation of the polarization directions. As a result, it is possible to extract the part including the anisotropic body 53 regardless of a direction or optical characteristics of the anisotropic body 53.

Alternatively, for example, it is also possible to set the predetermined threshold in accordance with optical characteristics or the like of an anisotropic body 53 of interest. In this case, the threshold is set on the basis of luminance differences or the like expected to be occurred in the target anisotropic body 53. This makes it possible to selectively extract the desired anisotropic body 53. In addition, a method or the like of setting the predetermined threshold is not limited. It is possible to use any threshold.

The analysis section 42 generates an emphasis image in which the extracted part is emphasized. In the emphasis image, for example, the extracted part (the part including the anisotropic body 53) is emphasized in comparison with the other parts. Alternatively, as the emphasis image, it is possible to generate an image that displays the extracted part only.

With regard to the emphasis image, a method or the like of emphasizing the extracted part is not limited. For example, it is possible to generate an emphasis image in which the extracted part is emphasized by prominent color such as red or green. Alternatively, it is possible to use any method of emphasizing the extracted part.

The generated emphasis image is output to the display unit 50 as an intraoperative image. The display unit 50 displays the intraoperative image. This makes it possible to easily distinguish whether or not the anisotropic body 53 is included in a surgery target part or an inspection target part, for example.

Note that, for example, in the case where an angle at which the minimum transmitted light intensity I(θ) is obtained is estimated and the first polarization direction is set to this angle in Step 102, it is possible to generate the first image signal at the rotation position of 0° and the second image signal at the rotation position of 45°, and end the loop process. In other words, two image signals having incident polarization angles θ that are different from each other by 45° are generated, and the loop process ends. This makes it possible to reduce an amount of time it takes to capture images.

Alternatively, it is possible to calculate a luminance difference between the first image signal and the second image signal each time a combination of image signals is generated in the case where the first and second polarization directions are rotated by the rotation angle ω=45°. Next, in the case where the luminance difference exceeds the predetermined threshold, it is considered that a combination of image signals that is sufficient to obtain adequate detection accuracy is acquired, and it is possible to end the loop process. This makes it possible to reduce an amount of time it takes to perform the process.

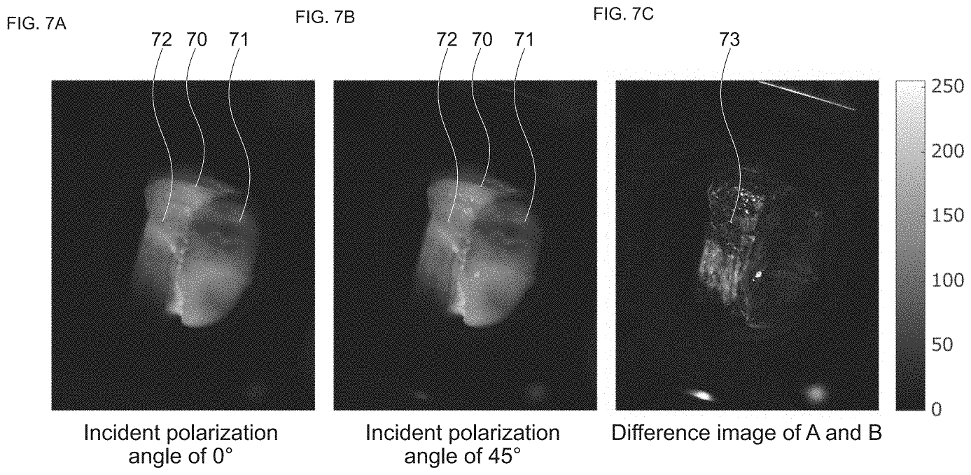

FIG. 7 is a diagram illustrating an example of images captured through the crossed nicols observation. An observation image A and an observation image B are illustrated on the left side and in the middle of FIG. 7. The observation image A and the observation image B shows images of the observation target 1 captured at the incident polarization angle θ of 0° and the incident polarization angle θ of 45°. In the example illustrated in FIG. 7, a sample 70 obtained by removing a part of a stomach of a pig is used as the observation target 1.

With regard to the sample 70, a part of a mucous membrane layer 71 (a right side of the sample) that is a surface layer part of lining of the stomach of the pig is removed, and a muscular layer 72 (a left side of the sample 70) positioned outside the mucous membrane layer 71 is exposed. During the observation, a white LED is used as the light source 21, and a color camera is used as the image sensor 32. The images illustrated in FIG. 7 are images obtained by converting the color images captured by the color camera into black and white images.

As shown in the observation images A and B, visibility of the sample 70 hardly changes even in the case where the incident polarization angle θ has changed. This is because components of reflection light scattered and reflected inside the sample 70 are larger than intensity of reflection light of the anisotropic body 53.

A difference image C generated on the basis of the observation images A and B is illustrated on the left side of FIG. 7. The difference image C is an image obtained by multiplying differences of RGB values of the respective pixels in the observation images A and B by 8. In the difference image C, reflection components of an optically anisotropic part 73 (the anisotropic body 53) are emphasized, but the other scattered/reflected components are offset. This makes it possible to determine that a bright part with large luminance differences is a part having large optical anisotropy.

As illustrated in the difference image C, most of a part corresponding to the muscular layer 72 are displayed brightly. In other words, it is understood that the muscular layer 72 includes many anisotropic bodies 53. On the other hand, a part corresponding to the mucous membrane layer 71 is substantially displayed in black. In other words, it is understood that the mucous membrane layer 71 does not include the anisotropic body 53.

As described above, it is possible to accurately detect the anisotropic body 53 having optical anisotropy by using the observation images (image signals) captured at angles different from each other by 45° in the crossed nicols observation.

Note that the emphasis image includes an image in which the luminance difference of the anisotropic body 53 is emphasized as it is like the difference image C. In other words, the difference image generated from the observation image captured at the incident polarization angle θ of 0° and the observation image captured at the incident polarization angle θ of 45° may be used as the intraoperative image. This makes it possible to reduce an amount of time it takes to perform an image process and the like, and this makes it possible to display an image with high response speed.

As described above, the endoscopic device 100 according to this embodiment irradiates the observation target 1 with the polarization light 3 of the first polarization direction. Among beams of reflection light 4 that are reflected by the observation target 1, the polarization components 5a and 5b of the second polarization direction that intersects with the first polarization direction are extracted. The first polarization direction and the second polarization direction are rotated while the intersection angle Φ is maintained, and an image signal of observation target 1 is generated on the basis of the extracted polarization components in accordance with the rotation operation. By using image signals generated through the rotation operation, it is possible to sufficiently support observation of the observation target 1.

As a method of observing a body tissue by emitting polarized light, there is a method using a Mueller imaging system capable of calculating a Mueller matrix. The Mueller imaging system sequentially irradiates a subject with a plurality of beams of polarization light having different polarization states, and acquires polarization properties (a depolarization ratio, a polarization ratio of light, a phase difference, an orientation of the phase difference, an orientation of absorption, or optical rotation) corresponding to the anisotropic body 53. In the case where a 4×4 Mueller matrix is generated by using such a method, it is necessary to acquire at least 16 or more images and perform an analysis process on these images. Therefore, it takes time to capture the images and perform the analysis process, and there is a possibility that it takes time to display an image to a doctor.

When using the endoscopic device 100 according to this embodiment, the rotation control section 41 rotates the first and second polarization directions on the basis of the rotation angle ω=45° while maintaining the substantially crossed nicols relation. The image sensor 32 generates the first and second image signals in the first and second states. With regard to the first and second image signals, the incident polarization angles θ are different from each other by 45°. It is possible to accurately observe the observation target 1 on the basis of the two image signals.

In other words, it is possible to observe the observation target 1 by capturing an image of the observation target 1 twice, and it is possible to shorten time it takes to capture images. Even in the case where images are repeatedly captured at a predetermined imaging angle d to improve accuracy of the observation as described above, it is possible to perform the observation by capturing an image approximately five times, for example. This highly contributes to shorten a length of time.

In addition, the analysis section 42 calculates luminance differences and differences in RGB values on the basis of image signals (the first and second image signals) captured at the incident polarization angles θ of approximately 0° and 45°. Accordingly, it is possible to accurately detect the anisotropic body 53 having optical anisotropy. In addition, the analysis section 42 is capable of easily detecting the anisotropic body 53 by comparing the first image signal with the second image signal, and this makes it possible to do the analysis in a short time.

The analysis section 42 generates the emphasis image in which the anisotropic body 53 is emphasized, and outputs it to the display unit as the intraoperative image. This makes it possible for a doctor to easily recognize presence/absence of the anisotropic body 53 on the basis of the intraoperative image, and this makes it possible to sufficiently support observation of the body tissue. In addition, it takes a short time to capture images and do analysis. Accordingly, for example, it is possible to display the intraoperative image in almost real time.

For example, to remove a tumor from a stomach or a large intestine, endoscopic submucosal dissection (ESD) has widely been performed in a field of gastroenterological medicine. When carrying out this surgical procedure, a doctor marks a resection area including a site of lesion, injects an agent such as saline into a submucosal layer, and removes the site of lesion that expands to the submucosal layer. Examples of a procedural accident of this surgical procedure include a perforation penetrating the muscular layer. For example, an incidence rate of the perforation during ESD of a stomach or a large intestine is several percent. There are problems of a surgical indication of the perforation and a postoperative complication.

The endoscopic device 100 according to this embodiment is capable of observing surgical removal of a tumor or the like through the ESD in almost real time, for example. Therefore, it is possible to recognize the risk of perforation by immediately detecting the anisotropic body 53 in the case where the anisotropic body 53 of the muscular layer or the like is exposed, for example. In addition, it is also possible to support diagnosis by detecting the exposure of the muscular layer or the like in the case of checking damage of the muscular layer after the removal. Of course, the surgical procedure is not limited to the ESD. The present technology is applicable to a surgical procedure or the like such as endoscopic mucosal resection (EMR).

In addition, for example, sometimes an optically anisotropic tissue such as collagen fibers may be exposed on a surface layer of a body tissue. In such a case, the crossed nicols observation using the endoscopic device 100 makes it possible to display differences in the optical anisotropy between the body tissues in a discriminable way. This makes it possible to identify a surgery target part, and observe a progression level or an area of disease, and this makes it possible to sufficiently support observation of the body tissue.

In addition, by using the endoscopic device 100, it is possible to detect heterogeneity of a fiber structure in a same structure. In other words, it is possible to detect a part or the like whose luminance difference has changed rapidly in a tissue having uniform luminance differences (change in transmitted light intensity I(θ)) between the first and second images. Therefore, it is possible to support a doctor to specify a broken part in the case where the fiber structure of the anisotropic body 53 such as a meniscus is broken, for example.

### Second Embodiment

Next, an information processing device according to a second embodiment of the present technology will be described. Hereinafter, description will be omitted with regard to structural elements and operation that are similar to the endoscopic device 100 described in the above embodiment.

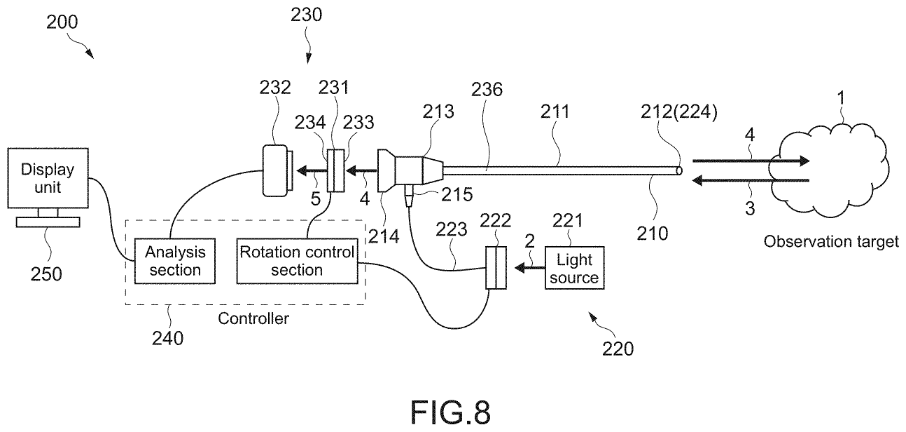

FIG. 8 is a diagram schematically illustrating a configuration example of an endoscopic device 200 that is an imaging device according to a second embodiment of the present technology. The endoscopic device 200 includes an insertion unit 210, an illumination system 220, an imaging system 230, a controller 240, and a display unit 250. The endoscopic device 200 is configured as a rigid endoscope that is used for a laparoscopic surgery or observation or the like of an otolaryngological area. Note that the controller 240 and the display unit 250 illustrated in FIG. 8 are configured in a way similar to the controller 40 and the display unit 50 illustrated in FIG. 1.

The insertion unit 210 includes a rigid section 211, a tip section 212, and an operation section 213. The rigid section 211 is configured as a thin tube, and includes hard material such as stainless. The material, size, and the like of the rigid section 211 are not limited. They may be appropriately set in accordance with its use purpose such as a surgery or observation.

The tip section 212 is provided at one end of the rigid section 211. The tip section 212 is inserted into an opening or the like made in an abdomen of a patient, and the tip section 212 reaches the vicinity of the observation target 1. Although not illustrated, the tip section 212 has an illumination opening, and an imaging opening. In addition, the tip section 212 may be appropriately provided with a nozzle or the like that is an outlet of water, air, or the like, a treatment tool outlet through which forceps or the like moves in and out, or the like.

The operation section 213 is provided at an end of the rigid section 211 opposite to the tip section 212. The operation section 213 includes a scope holder 214 and an optical port 215. A forceps port through which a treatment tool such as forceps moves in and out or the like may also function as the optical port 215, for example. In addition, the operation section 213 may be provided with a lever, a switch, or the like that is necessary to operate the insertion unit 210.

The illumination system 220 includes a light source 221, a first polarization element 222, a polarization maintaining fiber 223, and an illumination lens 224. The light source 221 and the first polarization element 222 are configured in ways similar to the light source 21 and the first polarization element 22 illustrated in FIG. 1. The polarization maintaining fiber 223 is inserted into the optical port 215 from the first polarization element 222, passes through the inside of the rigid section 211, and extends to the tip section 212. The illumination lens 224 is disposed in the illumination opening made in the tip section 212.

In the illumination system 220, the first polarization element 222 polarizes the illumination light 2 emitted from the light source 221 in the first polarization direction, and emits the polarized light to the observation target 1 via the polarization maintaining fiber 223 and the illumination lens 224.

The imaging system 230 includes a relay optical system 236, a second polarization element 231 and an image sensor 232. The relay optical system 236 is an optical system that connects the imaging opening to the scope holder 214, and is installed in the insertion unit 210. The relay optical system 236 is appropriately configured to be capable of maintaining a polarization direction of the reflection light 4. As illustrated in FIG. 8, the reflection light 4 reflected by the observation target 1 passes through the relay optical system 236 disposed in the insertion unit 210, and then is emitted.

The second polarization element 231 is disposed outside the scope holder 214. A liquid crystal polarizer including a liquid crystal variable wave plate 233 and a polarizing plate 234 is used as the second polarization element 231. As illustrated in FIG. 8, in the second polarization element 231, the liquid crystal variable wave plate 233 is disposed in a manner that the liquid crystal variable wave plate 233 faces the scope holder 214.

the reflection light 4 that has emitted from the observation target 1 and passed through the relay optical system 236 is incident on the liquid crystal variable wave plate 233. The second polarization element 231 extracts a polarization component 5 of the second polarization direction from beams of the reflection light 4, and the polarizing plate 234 emits the extracted polarization component 5.

The image sensor 232 is disposed across the second polarization element 231 from the scope holder 214. Therefore, the polarization component 5 of the second polarization direction extracted by the second polarization element 231 is incident on the image sensor 232.

In a way similar to the first embodiment, the endoscopic device 200 controls the first polarization element 222 and the second polarization element 231 and performs the crossed nicols observation (substantially crossed nicols observation). In other words, the first and second polarization directions are rotated while the crossed nicols (substantially crossed nicols) relation is maintained, and the image sensor 232 generates a first and second image signals. On the basis of the generated first and second image signals, a part or the like having high luminance differences is detected as an anisotropic body. Accordingly, it is possible to accurately detect the anisotropic body included in the observation target 1.

As described above, it is possible to perform the substantially crossed nicols observation even when using the endoscopic device 200 configured as the rigid endoscope. Therefore, it is possible to accurately detect the body tissue. This makes it possible to sufficiently support a doctor or the like to observe a body tissue not only in the case where an area of gastroenterological medicine is observed by using a soft endoscope, but also in the case of laparoscopic surgery or observation or the like of an otolaryngological area.

### Another Embodiment

The present technology is not limited to the above-described embodiments. Various other embodiments can be achieved.

In the above-described embodiments, the white LED or the like capable of emitting non-polarized illumination light is used as a light source. The present technology is not limited thereto. It is also possible to use a light source that emits illumination light in a predetermined polarization state such as linearly polarized light.

For example, as the light source, it is possible to use a laser light source such as a laser diode (LD). In this case, for example, illumination light is linearly polarized light of a predetermined polarization direction. For example, as the laser light source, it is possible to use a solid light source capable of emitting laser light of a desired wavelength or desired intensity. In addition, it is possible to synthesize white light by using beams of laser light emitted from a plurality of laser light sources. In addition, a specific configuration of the laser light source is not limited.

In the case where the illumination light is linearly polarized light, the liquid crystal variable wave plate is used as the first polarization element. For example, it is possible to use an element obtained by removing the polarizing plate 25 from the first polarization element 22 (liquid crystal polarizer) illustrated in FIG. 1. This makes it possible to rotate the polarization directions in any direction without reducing an amount of illumination light. In other words, it is possible to rotate a polarization direction of the illumination light to a desired direction and generate polarization light of the first polarization direction. As a result, it is possible to maintain high intensity of the polarization light to be emitted to the observation target, and it is possible to generate a bright observation image or the like.

Alternatively, for example, as the first polarization element, it is possible to use a ½λ plate (half wave plate) that is configured to be rotatable. For example, by appropriately rotating the ½λ plate, it is possible to rotate only the polarization direction to a desired direction with very little reduction in an amount of laser light. The specific configuration of the first polarization element is not limited. The first polarization element is appropriately configured in accordance with a wavelength or the like of the laser light.

In the above-described embodiments, the endoscopic devices 100 and 200 are configured as the observation devices. However, the observation device is not limited thereto. The observation device may be configured in a way different from the above-described embodiments. For example, a surgical microscope may be configured as the observation device. In other words, the surgical microscope including the first polarization element and the second polarization element may be appropriately configured. For example, it is possible to accurately detect an optically anisotropic body tissue (an anisotropic body) by controlling rotation of the first and second polarization directions through the process illustrated in FIG. 6. This makes it possible to magnify and observe the anisotropic body, for example.

Each of the first polarization element and the second polarization element may be configured to be attachable and detachable. For example, holder mechanisms of holding the first and second polarization elements or the like may be configured as detachable units. For example, in the case where the body tissue using polarization light is not performed, it is possible to detach the first and second polarization elements and observe the body tissue or the like. By detaching each of the polarization elements, it is possible to carry out observation in a bright field without polarizing illumination light, for example. This makes it possible to carry out observation in accordance with types or the like of body tissues, and this results in improvement in convenience.

The rotation control section is capable of setting the first polarization direction and the second polarization direction to be substantially parallel to each other. In other words, the rotation control section is capable of controlling the first polarization element, the second polarization element, and the like in a manner that the first polarization direction and the second polarization direction achieve the parallel nicols (substantially parallel nicols) relation.

By setting the first polarization direction to be substantially parallel to the second polarization direction, it is possible to brightly display the entire body tissue. Therefore, for example, it is possible to switch visibility of the body tissue in accordance with situations. This makes it possible for a doctor to select a desired observation method in accordance with progress or the like of a surgery, and this makes it possible to sufficiently support observation of the body tissue.

In addition, when a computer operated by the doctor or the like and another computer capable of communication via a network work in conjunction with each other, the imaging method according to the present technology is executed, and this makes it possible to configure the imaging device according to the present technology.

That is, the imaging method according to the present technology can be executed not only in a computer system consisting of a single computer, but also in a computer system in which a plurality of computers cooperatively operates. It should be noted that in the present disclosure, the system means an aggregate of a plurality of components (devices, modules (parts), or the like) and it does not matter whether or not all the components are housed in a same casing. Therefore, a plurality of devices housed in separate casings and connected to one another via a network is treated as a system, and a single device including a plurality of modules housed in a single casing is also treated as a system.

The execution of the imaging method according to the present technology by the computer system includes, for example, both of a case where control of rotation of the first and second polarization directions, generation of image signals depending on rotation operation of the first and second polarization directions, and the like are executed by a single computer and a case where those processes are executed by different computers. Further, the execution of the respective processes by predetermined computers includes causing another computer to perform some or all of those processes and acquiring results thereof.

That is, the imaging method according to the present technology are also applicable to a cloud computing configuration in which one function is shared and cooperatively processed by a plurality of devices via a network.

In addition, the present technology is applicable to observation devices and observation systems not only in medical/biological fields but also in various kinds of other fields.

Out of the feature parts according to the present technology described above, at least two feature parts can be combined. That is, the various feature parts described in the embodiments may be arbitrarily combined irrespective of the embodiments. Further, various effects described above are merely examples and are not limited, and other effects may be exerted.

Note that the present technology may also be configured as below.

(1) An imaging device including:

a first polarization section that irradiates a body tissue with polarization light of a first polarization direction;

a second polarization section that extracts a polarization component of a second polarization direction that intersects with the first polarization direction, from beams of reflection light that are the polarization light reflected by the body tissue;

a rotation control section that rotates each of the first polarization direction and the second polarization direction while maintaining an intersection angle between the first polarization direction and the second polarization direction; and

a generation section that generates an image signal of the body tissue on the basis of the polarization component of the reflection light extracted by the second polarization section in accordance with rotation operation performed by the rotation control section.

(2) The imaging device according to (1), in which

the first polarization section includes a first polarization element that polarizes at least part of illumination light emitted from a light source, in the first polarization direction, and

the second polarization section includes a second polarization element that extracts the polarization component of the second polarization direction.

(3) The imaging device according to 81) or (2),

in which the intersection angle is an angle in a range of 90°±2°.

(4) The imaging device according to any one of (1) to (3),

in which the rotation control section rotates the first polarization direction and the second polarization direction in synchronization with each other.

(5) The imaging device according to any one of (1) to (4),

in which the generation section generates a first image signal in the case where each of the first polarization direction and the second polarization direction is in a predetermined state, and generates a second image signal in the case where the rotation control section rotates each of the first polarization direction and the second polarization direction in the predetermined state by a predetermined angle.

(6) The imaging device according to (5),

in which the predetermined angle is an angle in a range of 45°±22.5°.

(7) The imaging device according to (5) or (6), further including

an analysis section that analyzes each of the first image signal and the second image signal.

(8) The imaging device according to (7), in which

the first image signal includes a plurality of first pixel signals each including luminance information,

the second image signal includes a plurality of second pixel signals each including luminance information, and

the analysis section calculates luminance differences between the first pixel signals and the second pixels signals.

(9) The imaging device according to (8),

in which the analysis section extracts a part having the luminance difference higher than a predetermined threshold.

(10) The imaging device according to (9),

in which the analysis section generates an emphasis image in which the extracted part is emphasized.

(11) The imaging device according to (10),

in which the analysis section outputs the emphasis image as an intraoperative image.

(12) The imaging device according to any one of (2) to (11),

in which each of the first polarization element and the second polarization element is configured to be attachable and detachable.

(13) The imaging device according to any one of (1) to (12), which is configured as an endoscope or a microscope.
(14) The imaging device according to any one of (1) to (13),

in which the rotation control section is capable of setting the first polarization direction and the second polarization direction to be substantially parallel to each other.

(15) An imaging method, including

by a computer system,

irradiating a body tissue with polarization light of a first polarization direction;

extracting a polarization component of a second polarization direction that intersects with the first polarization direction, from beams of reflection light that are the polarization light reflected by the body tissue;

rotating each of the first polarization direction and the second polarization direction while maintaining an intersection angle between the first polarization direction and the second polarization direction; and

generating an image signal of the body tissue on the basis of the extracted polarization component of the reflection light in accordance with rotation operation performed by the rotation control section.

### REFERENCE SIGNS LIST

Φ intersection angle
ω rotation angle
1 observation target
2 illumination light
3 polarization light
4, 4a, 4b, 4c reflection light
5, 5a, 5b polarization component
20, 220 illumination system
21, 221 light source
22, 222 first polarization element
31, 231 second polarization element
32, 232 image sensor
41 rotation control section
42 analysis section
50, 250 display unit
53 anisotropic body
100, 200 endoscopic device

1. An imaging device comprising:
a first polarization section that irradiates a body tissue with polarization light of a first polarization direction;
a second polarization section that extracts a polarization component of a second polarization direction that intersects with the first polarization direction, from beams of reflection light that are the polarization light reflected by the body tissue;
a rotation control section that rotates each of the first polarization direction and the second polarization direction while maintaining an intersection angle between the first polarization direction and the second polarization direction; and
a generation section that generates an image signal of the body tissue on a basis of the polarization component of the reflection light extracted by the second polarization section in accordance with rotation operation performed by the rotation control section.

2. The imaging device according to claim 1, wherein
the first polarization section includes a first polarization element that polarizes at least part of illumination light emitted from a light source, in the first polarization direction, and
the second polarization section includes a second polarization element that extracts the polarization component of the second polarization direction.

3. The imaging device according to claim 1, wherein
the intersection angle is an angle in a range of 90°±2°.

4. The imaging device according to claim 1, wherein
the rotation control section rotates the first polarization direction and the second polarization direction in synchronization with each other.

5. The imaging device according to claim 1, wherein
the generation section generates a first image signal in a case where each of the first polarization direction and the second polarization direction is in a predetermined state, and generates a second image signal in a case where the rotation control section rotates each of the first polarization direction and the second polarization direction in the predetermined state by a predetermined angle.

6. The imaging device according to claim 5, wherein
the predetermined angle is an angle in a range of 45°±22.5°.

7. The imaging device according to claim 5, further comprising
an analysis section that analyzes each of the first image signal and the second image signal.

8. The imaging device according to claim 7, wherein
the first image signal includes a plurality of first pixel signals each including luminance information,
the second image signal includes a plurality of second pixel signals each including luminance information, and
the analysis section calculates luminance differences between the first pixel signals and the second pixels signals.

9. The imaging device according to claim 8, wherein
the analysis section extracts a part having the luminance difference higher than a predetermined threshold.

10. The imaging device according to claim 9, wherein
the analysis section generates an emphasis image in which the extracted part is emphasized.

11. The imaging device according to claim 10, wherein
the analysis section outputs the emphasis image as an intraoperative image.

12. The imaging device according to claim 2, wherein
each of the first polarization element and the second polarization element is configured to be attachable and detachable.

13. The imaging device according to claim 1, which is configured as an endoscope or a microscope.

14. The imaging device according to claim 1, wherein
the rotation control section is capable of setting the first polarization direction and the second polarization direction to be substantially parallel to each other.

15. An imaging method, comprising:
by computer system,
irradiating a body tissue with polarization light of a first polarization direction;
extracting a polarization component of a second polarization direction that intersects with the first polarization direction, from beams of reflection light that are the polarization light reflected by the body tissue;
rotating each of the first polarization direction and the second polarization direction while maintaining an intersection angle between the first polarization direction and the second polarization direction; and
generating an image signal of the body tissue on a basis of the extracted polarization component of the reflection light in accordance with rotation operation performed by the rotation control section.
